(19) United States
(12) Patent Application Publication
(10) Pub. No.: US 2008/0147086 A1
(43) Pub. Date: Jun. 19, 2008

(54) Integrating 3D images into interventional procedures

(76) Inventors: Marcus Pfister, Bubenreuth (DE); Michael Maschke, Lonnerstadt (DE); Jan Boese, Eckental (DE); Norbert Rahn, Forchheim (DE)

Correspondence Address: BRINKS HOFER GILSON & LIONE, P.O. BOX 10395, CHICAGO, IL, 60610, US

(21) Appl. No.: 11/544,846

(22) Filed: Oct. 5, 2006

(51) Int. Cl. A61B 19/00

(52) U.S. Cl. 606/130

(57) ABSTRACT

Three-dimensional image datasets are used to assist in the visualization of an interventional procedure. The three-dimensional image datasets are registered to two-dimensional images acquired by a medical imaging device. A display device can display a fusion visualization of the three-dimensional image datasets and the two-dimensional image. A monitoring device can monitor the progress of a medical instrument used in the interventional procedure. A processor can incorporate the position of the medical instrument in the fusion visualization displayed by the display device.

### BACKGROUND OF THE INVENTION

1. Technical Field

The present embodiments relate to integrating three-dimensional images into interventional procedures. In particular, acquired two- and three-dimensional image datasets are processed and displayed as a fusion visualization during an interventional procedure.

2. Related Art

Interventional procedures involving a minimal amount of invasiveness for patients are increasingly prevalent. Examples of minimally invasive interventional procedures include cardiac valve replacement or repair, stem cell therapy, the placement of balloon appellation devices, tumor treatment, spinal procedures, and invasive joint therapy. Other examples of interventional procedures include vertebroplasty, kyphoplasty, myelography, bone biopsy, discography, intradiscal electrotherma therapy, and perpiradicular therapy. The medical instruments used in these interventional procedures typically include catheters, needles, and guidewires, which are often introduced into an organ cavity or portion of the patient undergoing the interventional procedure. These interventional procedures are typically monitored using a medical imaging device capable of acquiring two-dimensional images, and a doctor or technician can use the acquired two-dimensional images to monitor the medical instrument being used. Examples of acquired two-dimensional images include fluoroscopic images, computed tomography images, magnetic resonance images, ultrasound and positron emission tracking images.

Although the medical instrument can be monitored using the acquired two-dimensional images, the anatomy of the patient undergoing the interventional procedure is often inadequately displayed in these two-dimensional images. Hence, the doctor or the technician is unable to monitor the medical instrument and its position as they relate to the anatomy of the patient.

### SUMMARY

By way of introduction, the embodiments described below include a system and a method for integrating three-dimensional images into interventional procedures. The system is operative to acquire and display images during an interventional procedure. The system includes a medical imaging device, a monitoring device, a processor, and a display device. The medical imaging device can acquire two-dimensional images of the organ cavity or portion of the patient undergoing the interventional procedure. The monitoring device can monitor the patient and can detect changes in the patient's position or alignment. The monitoring device can also monitor the organ cavity of the patient. The monitoring device can further be configured to monitor the medical instrument used in the interventional procedure. A processor is coupled with the monitoring device and the medical imaging device. The processor can generate a 3-D/2-D fusion visualization of the organ cavity or portion of the patient based on an acquired two-dimensional image and a three-dimensional image dataset. The display device can then display the 3-D/2-D fusion visualization.

The method involves displaying an interventional procedure using three-dimensional image datasets. The method includes acquiring a three-dimensional image dataset and a two-dimensional image. The three-dimensional image dataset is then registered to the two-dimensional image. The three-dimensional image dataset and the two-dimensional image are then displayed as a 3-D/2-D fusion visualization. The three-dimensional image dataset may be displayed as a separate three-dimensional image separate from a display of the two-dimensional image.

The present invention is defined by the following claims, and nothing in this section should be taken as a limitation on those claims. Further aspects and advantages of the embodiments are discussed below in conjunction with the preferred embodiments and may be later claimed independently or in combination.

### BRIEF DESCRIPTION OF THE DRAWINGS

The system may be better understood with reference to the following drawings and description. The components in the figures are not necessarily to scale, emphasis instead being placed upon illustrating the principles of the invention. Moreover, in the figures, like referenced numerals designate corresponding parts throughout the different views.

FIG. 1 is a block diagram of one embodiment of a system for acquiring images and displaying an interventional procedure.

FIG. 2 is a block diagram illustrating the fusion visualization of a three-dimensional image and a two-dimensional image.

FIG. 3 is a flow chart diagram of one embodiment of a method for displaying an interventional procedure.

FIG. 4 is a flow chart diagram of one embodiment of a method for registering a three-dimensional image dataset.

FIG. 5 is a flow chart diagram of one embodiment of a method for performing an interventional procedure.

### DETAILED DESCRIPTION

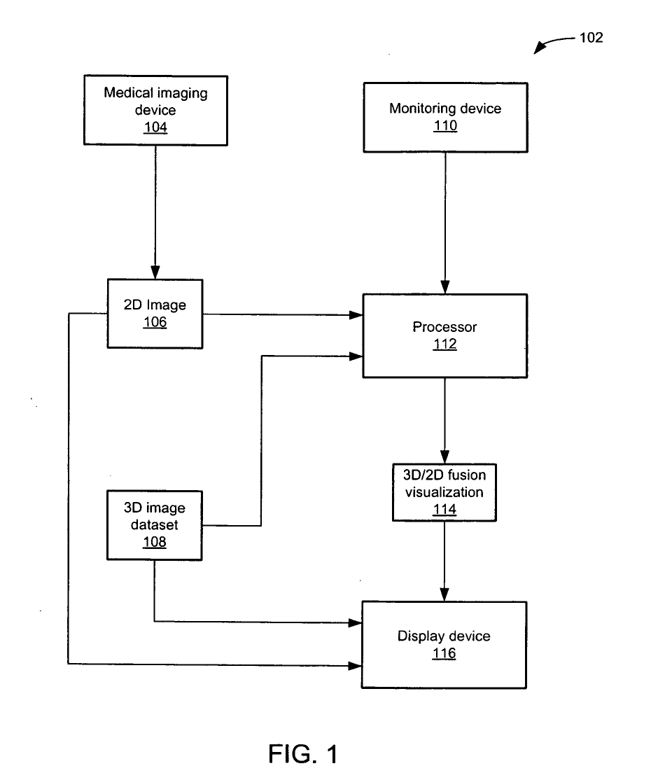

FIG. 1 shows one embodiment of a system 102 for acquiring and displaying during an interventional procedure using a three-dimensional image dataset registered to a two-dimensional image. The system 102 includes a medical imaging device 104 that can acquire a two-dimensional image 106. The system 102 also includes a monitoring device 110 coupled to a processor 112. In one embodiment, the processor 112 receives as input the two-dimensional image 106 and a three-dimensional image dataset 108. The processor 112 is operative to produce a fusion visualization 114 of the two-dimensional image 106 and the three-dimensional image dataset 108. The 3-D/2-D fusion visualization image 114 may then be displayed using a display device 116. The display device 116 can also receive as input the three-dimensional image dataset 108 and the two-dimensional image 106 for displaying separately.

The medical imaging device 104 is a medical imaging device operative to generate two-dimensional images, such as fluoroscopic images, angiographic images, ultrasound images, X-ray images, any other now known or later developed two-dimensional image acquisition technique, or combinations thereof. For example, in one embodiment the medical imaging device 104 is an X-ray imaging device, such as the ARCADIS Orbic C-arm imaging device available from Siemens Medical Solutions of Siemens AG headquartered in Malvern, Pa. In another embodiment, the medical imaging device 104 is an operation microscope, such as the OMS-610 Operation Microscope available from Topcon America Corporation headquartered in Paramus, N.J. In yet another embodiment, the medical imaging device 104 is an imaging device capable of producing fluoroscopic images, such as the AXIOM Iconos R200 also available from Siemens Medical Solutions of Siemens AG. The medical imaging device 104 may also be an imaging device capable of producing angiographic images, such as the AXIOM Artis dTA also available from Siemens Medical Solutions of Siemens AG.

The two-dimensional image 106 acquired by the medical imaging device 104 may be a fluoroscopic image, an angiographic image, an x-ray image, an ultrasound image, any other two-dimensional medical image, or combination thereof. For example, the two-dimensional image 106 may be acquired using computed tomography (CT), magnetic resonance imaging (MRI), positron emission tomography (PET), single photon emission computed tomography (SPECT), any other two-dimensional image technique now known or later developed, or combinations thereof. The two-dimensional image may be a two-dimensional image of a scanned organ cavity or a portion of the patient undergoing the interventional procedure. For example, the two-dimensional image 106 may be an x-ray image of the patient's chest cavity. In another embodiment, the two-dimensional image 106 may be a fluoroscopic image of the patient's gastrointestinal tract.

The three-dimensional image dataset 108 is a dataset representative of an organ cavity or portion of the patient registered to the two-dimensional image 106 produced by the medical imaging device 104. The three-dimensional image dataset 108 may be acquired using any three dimensional technique, including pre-operative techniques, intra-operative techniques, fused 3-D volume imaging techniques, any other now known or later developed techniques, or combinations thereof. Examples of pre-operative techniques include, but are not limited to, computed tomography (CT), magnetic resonance imaging (MRI), positron emission tomography (PET), single photon emission computed tomography (SPECT), ultrasound or combinations thereof. Examples of intra-operative techniques include, but are not limited to, 3D digital subtraction angiography, 3D digital angiography, rotational angiography, such as the DynaCT technique developed by Siemens Medical Solutions of Siemens AG, 3D ultrasound, or combinations thereof. Examples of fused 3-D volume imaging techniques include, but are not limited to, the PET/CT imaging technique and the SPECT+CT imaging technique, both developed by Siemens Medical Solutions of Siemens AG. Other types of three-dimensional imaging techniques now known or later developed are also contemplated.

The three-dimensional image dataset 108 is registered to the two-dimensional image 106. Registration generally refers to the spatial modification (e.g., translation, rotation, scaling, deformation) or known spatial relationship of one image relative to another image in order to arrive at an ideal matching of both images. Registration techniques include, but are not limited to, registration based on calibration information of the medical imaging device, feature-based registration, speckle based registration, motion tracking, intensity-based registration, implicit registration, and combinations thereof. Further registration techniques are explained in Chapter 4 of Imaging Systems for Medical Diagnostics (2005) by Arnulf Oppelt.

The monitoring device 110 monitors the interventional procedure of the system 102. In one embodiment, the monitoring device 110 is a camera located on the medical imaging device 104 that provides real-time images of the organ cavity or portion of the patient to the processor 112 for display on the display device 116. In another embodiment, the monitoring device 110 is an instrument localization device used to locate the medical instrument in the organ cavity or portion of the patient. For example, the instrument localization device may use magnetic tracking to track the location of the medical instrument in the organ cavity or portion of the patient. The instrument localization device can provide the coordinates of the medical instrument within the organ cavity or portion of the human patient to the processor 112 for later displaying on the display device 116. In this example, the three-dimensional image dataset 108 may also be registered to the instrument localization device. In another embodiment, the monitoring device 110 is a magnetic navigation device operative to manipulate the medical instrument being used in the organ cavity or portion of the human patient. The magnetic navigation device can provide the coordinates of the medical instrument in the organ cavity or portion of the patient to the processor 112 for later displaying on the display device 116. In this embodiment, the three-dimensional image dataset 108 can also be registered to the instrument navigation device.

The processor 112 is a general processor, a data signal processor, graphics card, graphics chip, personal computer, motherboard, memories, buffers, scan converters, filters, interpolators, field programmable gate array, application-specific integrated circuit, analog circuits, digital circuits, combinations thereof, or any other now known or later developed device for generating a fusion visualization of the two-dimensional image 106 and the three-dimensional image dataset 108. The processor 112 includes software or hardware for rendering a three-dimensional representation of the three dimensional image dataset 108, such as through alpha blending, minimum intensity projection, maximum intensity production, surface rendering, or other now known or later developed rendering technique. The processor 112 also has software for visualizing the fusion of the two-dimensional image 106 with the three-dimensional image dataset 108. The resulting 3-D/2-D fusion visualization 114 produced by the processor 112 is then transmitted to the display device 116. The term fusion visualization generally refers to the display of the two-dimensional image 106 and the three-dimensional image dataset 108 in a manner relating to their current registration. Fusion visualization techniques include, but are not limited to, intensity-based visualization, volume rendering technique, digitally reconstructed radiographs, overlaying graphic primitives, back projection, subtracted visualization, or combinations thereof. The processor 112 may also be configured to incorporate the medical measurement monitored by the monitoring device 110 in the 3-D/2-D fusion visualization 114 based on the coordinates of the medical instrument provided by the monitoring device 110. The processor 112 may also be configured to update the position and orientation of the medical instrument relative to the three-dimensional image dataset 108 and the two-dimensional image 106 based on the output provided by the monitoring device 110.

The display device 116 is a monitor, CRT, LCD, plasma screen, flat-panel, projector are other now known or later developed display device. The display device 116 is operable to generate images of the 3-D/2-D fusion visualization 114 produced by the processor 112. The display device 116 is also operable to display a separate three-dimensional image of the three-dimensional image dataset 108 and to display the two-dimensional image 106 provided by the medical imaging device 104. The display device 116 can also be configured to display the medical instrument monitored by the monitoring device 110.

The system 102 may further include a user input for manipulating the medical imaging device 104, the monitoring device 110, the processor 112, the display device 116, or combination thereof. The user input could be a keyboard, touchscreen, mouse, trackball, touchpad, dials, knobs, sliders, buttons, or combinations thereof or other now known or later developed user input devices.

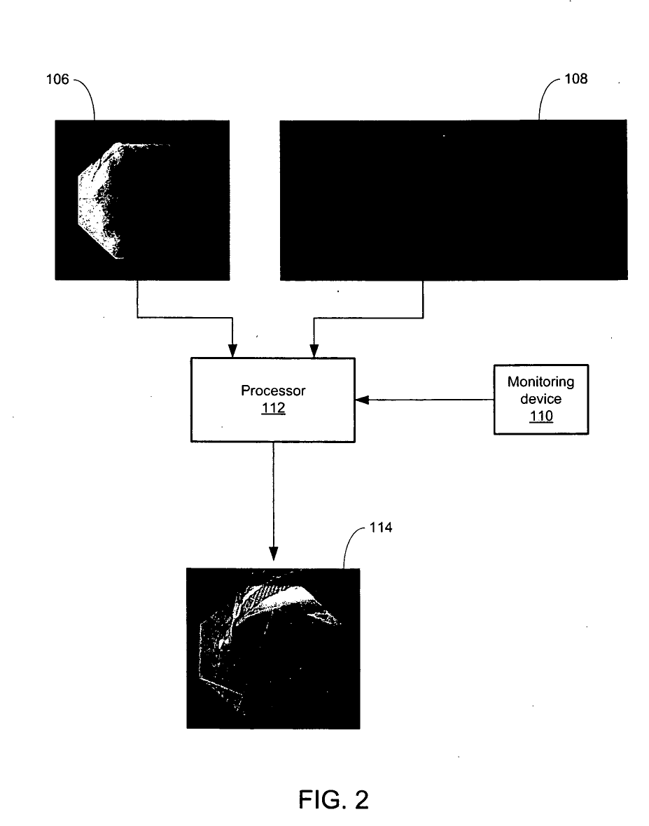

FIG. 2 is a block diagram illustrating the 3-D/2-D fusion visualization 114 of the two-dimensional image 106 and the three-dimensional image dataset 108. The three dimensional image dataset 108 is registered to the two-dimensional image 106. The processor 112 receives the three dimensional image dataset 108 and the two-dimensional image 106 to produce the 3-D/2-D fusion visualization 114 of the two-dimensional image 106 and the three-dimensional image dataset 108. The monitoring device 110 can also provide coordinates to the processor 112 of the medical instrument being used in the organ cavity represented by the three-dimensional image dataset 108 and the two-dimensional image 106. The processor 112 uses the coordinates from the monitoring device 110 to incorporate the position of the medical instrument in the 3-D/2-D fusion visualization 114. The monitoring device 110 may be further configured to provide the coordinates to the processor 112 in real-time during the interventional procedure. Further two-dimensional images and three-dimensional image datasets can be provided to the processor 112 for updating the 3-D/2-D fusion visualization 114 produced by the processor 112.

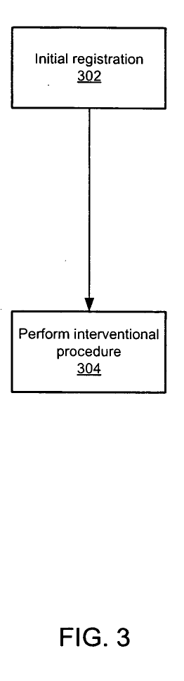

FIG. 3 is a flow chart diagram of one embodiment of a method for displaying an interventional procedure using a three-dimensional image dataset 108 registered to a two-dimensional image 106. As shown in FIG. 3, an initial registration of the three-dimensional image dataset 108 to the two-dimensional image 106 (Block 302) occurs before the interventional procedure is performed (Block 304).

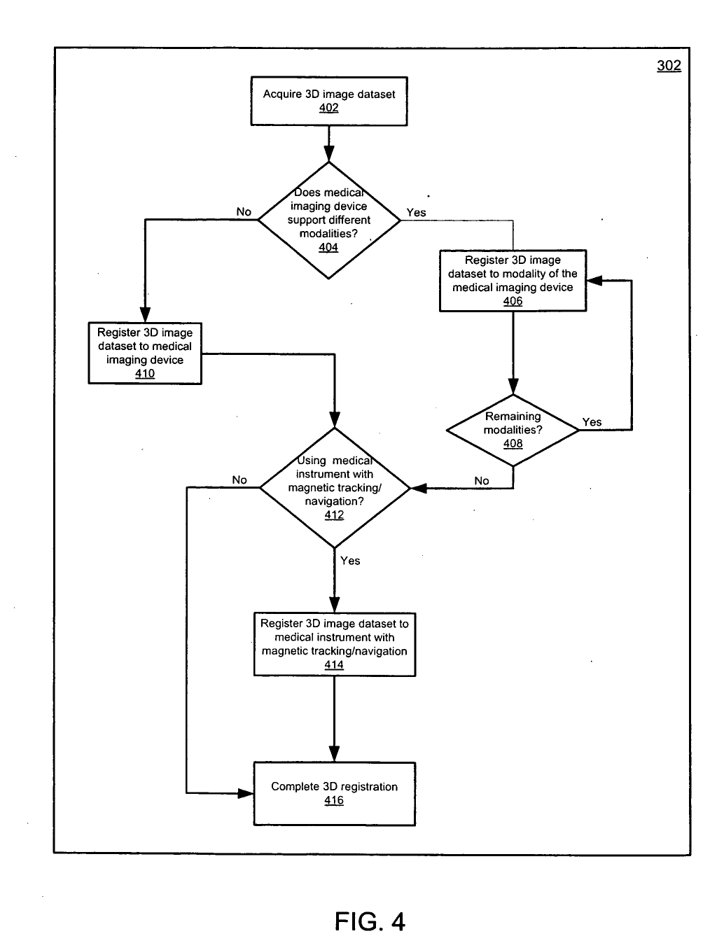

FIG. 4 is a flow chart diagram of one embodiment of a method for registering a three-dimensional image dataset. As shown in FIG. 4, the doctor or technician may first acquire the three-dimensional image dataset 108 of the organ cavity or portion of the patient in preparation for the interventional procedure (Block 402). In the embodiment shown in FIG. 4, the three-dimensional image dataset 108 is acquired using a pre-operative technique. For example, the three-dimensional image dataset 108 may be acquired using computed tomography, magnetic resonance imaging, positron emission tomography, single photon emission computed tomography, or any other now known or later developed three-dimensional image dataset acquisition technique. In another embodiment, the doctor or technician could acquire the three-dimensional image dataset 108 during the interventional procedure using an intra-operative technique such as 3D digital subtraction angiography, 3D digital angiography, DynaCT, or any other now known or later developed intra-operative technique, or combination thereof.

Once the three-dimensional image dataset 108 has been acquired (Block 402), the doctor or other technician may then determine whether the medical imaging device supports different modalities (Block 404). For example, the medical imaging device 104 may support multiple imaging modalities such as CT, MRI, PET, SPECT, any other now known or later developed imaging modality, or combinations thereof. If the doctor or the technician determines that the medical imaging device 104 only has one type of modality, such as CT, the doctor or the technician then registers the three-dimensional image dataset 108 to the one modality of the medical imaging device 104 (Block 410). The spatial relationship of the scanning devices determines the spatial relationship of the scanned regions. Since the two and three-dimensional image sets correspond to the scanned regions, the spatial relationships of the image sets is determined from the spatial relationship of the scanning devices. If the doctor or the technician determines that the medical imaging device 104 supports multiple modalities, the doctor or the technician then proceeds to register the three-dimensional image dataset 108 to the two-dimensional image 106 based on one or more of the various modalities supported by the medical imaging device 104 (Block 406). After each registration, the doctor or technician determines whether there are remaining modalities for the medical imaging device 104 (Block 408). If there are remaining modalities, the doctor or the technician can then proceed to register the three-dimensional image dataset 108 to the remaining one or more modalities (Block 406). Alternatively, or in addition to registering the three-dimensional image dataset 108 to the one or more imaging modalities of the medical imaging device 104, the three-dimensional image dataset 108 could be registered to the geometry of the medical imaging device 104.

Although FIG. 4 shows registration of the three-dimensional image dataset 108 to the based on the imaging modalities of the medical imaging device 104, other types of registration are also possible. For example, the three-dimensional image dataset 108 could be registered to the two-dimensional image 106 using image-based registration techniques, such as rigid and affine registration, geometry-based registration, visual alignment registration, feature-based registration, landmark-based registration, intensity-based registration, non-rigid registration, any other known or later developed registration technique, or combinations thereof.

The doctor or the technician determines whether the monitoring device 110 supports magnetic tracking or magnetic navigation for use during the interventional procedure (Block 412). If the monitoring device 110 does not support magnetic tracking and/or magnetic navigation, the doctor or the technician proceeds to complete the registration of the three-dimensional image dataset 108 to the medical imaging device 104 (Block 416). If the monitoring device 110 supports magnetic tracking and/or magnetic navigation, the doctor or the technician can register the three-dimensional image dataset 108 to the monitoring device 110 based on magnetic tracking and/or magnetic navigation (Block 414). Registering the three-dimensional image dataset 108 to the monitoring device 1 10 based on magnetic tracking and/or magnetic navigation (Block 414) may also include registering the three-dimensional image dataset 108 to the medical instrument being used in the interventional procedure. Alternatively, the monitoring device 110 is registered to the two-dimensional image.

The doctor or the technician proceeds to complete the registration process (Block 416). Completing the registration process may include modifying the registration of the three-dimensional image dataset 108, modifying the three-dimensional image dataset 108, or saving the three-dimensional image dataset 108 in memory of the system 102. Modifying the registration of the three-dimensional image dataset 108 or modifying the three-dimensional image dataset 108 may include adding to the three-dimensional image dataset 108, removing information from the three-dimensional image dataset 108, editing the three-dimensional image dataset 108, or combinations thereof.

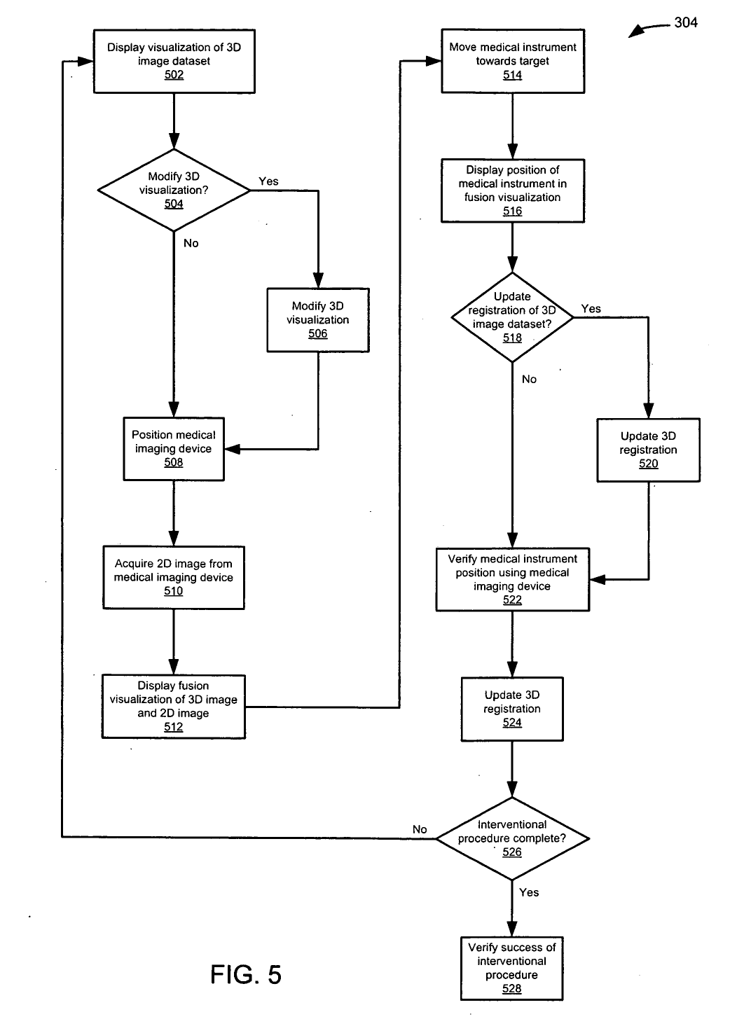

As shown in FIG. 3, after the registration process has completed (Block 302), the doctor or the technician performs the interventional procedure (Block 304). FIG. 5 is a flow chart diagram of one embodiment of a method for performing an interventional procedure using a three-dimensional image dataset registered to a two-dimensional image. In performing the interventional procedure, the doctor or the technician may first display a visualization of the three-dimensional image dataset 108 on the display device 116 (Block 502). The doctor or the technician may then decide to modify the visualization of the three-dimensional image dataset 108 (Block 504). If the doctor or the technician decides not to modify the visualization of the three-dimensional image dataset 108, the doctor or the technician then proceeds to position the medical imaging device 104 over or near the patient undergoing the interventional procedure (Block 508). If the doctor or the technician decides to modify the visualization of the three-dimensional image dataset 108, the doctor or the technician then modifies the visualization of the three-dimensional image dataset 108 (Blocked 506).

In one embodiment, the doctor or the technician modifies the visualization of the three-dimensional image dataset 108 by editing the visualization on an image processing workstation. In another embodiment, the doctor or the technician modifies the visualization of the three-dimensional image dataset 108 by changing the transfer function used to display the visualization of the three-dimensional image dataset 108. In yet another embodiment, the doctor or the technician modifies a visualization of the three-dimensional image dataset 108 by clipping the displayed visualization. Modifying the visualization of the three-dimensional image dataset 118 could also include changing the volume rendering mode used to display the visualization of the three-dimensional image dataset 108. In a further embodiment, the doctor or the technician modifies the visualization of the three-dimensional image dataset 108 by marking a target in the visualization, such as by marking bile ducts or a particular tumor for a biopsy. The doctor or the technician could modify the visualization of the three-dimensional image dataset 108 using any one of the aforementioned techniques or combination thereof.

After the doctor or the technician has finished modifying the visualization of the three-dimensional image dataset 108 (Block 506), or has decided not to modify the visualization of the three-dimensional image dataset 108 (Block. 504), the doctor or the technician then positions the medical imaging device 104 over or near the patient undergoing the interventional procedure to obtain a working projection (e.g., the two-dimensional image 106) (Block 508). In positioning the medical imaging device 104, the doctor or the technician may alter the rotational alignment of the medical imaging device 104, the directional alignment of the medical imaging device 104, the zoom factor used to acquire the two-dimensional image 106, any other similar or equivalent positioning alterations, or combinations thereof. In one embodiment, the processor 112 reregisters the three-dimensional image dataset 108 to the two-dimensional image 106 based on the geometry of the medical imaging device 104 according to the positioning alterations made to the medical imaging device 104. In another embodiment, the processor 112 does not reregister the three-dimensional image dataset 108 to the two-dimensional image 106 based on the geometry of the medical imaging device 104 after positioning medical imaging device 104, but instead later uses image based registration.

After the doctor or the technician has positioned the medical imaging device 104 over or near the patient undergoing the interventional procedure, the doctor or the technician then acquires the two-dimensional image 106 of the organ cavity or the portion of the patient using the medical imaging device 104 (Block 510). After the two-dimensional image 106 has been acquired (Block 510), the processor 112 then creates the 3-D/2-D fusion visualization 114 of the two-dimensional image 106 and the three-dimensional image dataset 108, which is then displayed on the display device 116 (Block 512).

While the 3-D/2-D fusion visualization 114 is displayed on the display device 116, the doctor or the technician may adjust a blending of the two-dimensional image 106 and the three-dimensional image generated from the three-dimensional image dataset 108. For example, the doctor or the technician may only want to see the two-dimensional image 106 of the 3-D/2-D fusion visualization 114. In this case, the doctor or the technician can adjust the blending so that only the two-dimensional image 106 is displayed on the display device 116. In another example, and doctor may only want to see the three dimensional image of the three-dimensional image dataset 108 in the 3-D/2-D fusion visualization 114. In this case the doctor or the technician can adjust the blending of the 3-D/2-D fusion visualization 114 such that only the three-dimensional image of the three-dimensional image dataset 108 is displayed. In an alternative embodiment, the display device 116 displays the two-dimensional image 106, the three-dimensional image representative of the three-dimensional image dataset 108, and the 3-D/2-D fusion visualization 114 output by the processor 112.

The 3-D/2-D fusion visualization 114 may be a fusion visualization produced using a blending, flexible a-blending, a volume rendering technique overlaid with a multiplanar reconstruction, a volume rendering technique overlaid with a maximum intensity projection, any other now known or later developed fusion visualization technique, or combinations thereof. In one embodiment, the 3-D/2-D fusion visualization 114 may be produced by displaying a visualization of the three-dimensional image dataset 108 rendered using a volume rendering technique overlaid with the previously registered two-dimensional image 106. For example, the three-dimensional image dataset 108 may be displayed using a volume rendering technique and the two-dimensional image 106 may be displayed as a maximum intensity projection overlaid on the rendered volume as a plane of the three-dimensional image dataset 108. In this example, the processor 112 could be operative to rotate the 3-D/2-D fusion visualization displayed by the display device 116 so as to provide a three-dimensional rotational view of the three-dimensional image dataset 108 and the two-dimensional image 106. In another embodiment, the 3-D/2-D fusion visualization 114 is displayed incorporating the medical instrument. For example, the two-dimensional image 106 may be acquired as a maximum intensity projection such that the medical instrument appears in the two-dimensional image 106. In this example, the 3-D/2-D fusion visualization 114 may be displayed as a visualization of the three-dimensional image dataset 108 rendered using a volume rendering technique and the two-dimensional image 106 may be displayed overlaid on the rendered volume as a plane of the three-dimensional image dataset 108 such that the medical instrument appears in the display of the 3-D/2-D fusion visualization 114.

Once or while the 3-D/2-D fusion visualization 114 is displayed (Block 512), the doctor or the technician then progresses the medical instrument towards the target of the interventional procedure (Block 514). The medical instrument the doctor or the technician uses may depend on the type of interventional procedure. For example, if the interventional procedure involves a tumor biopsy, bronchioscopy, or other similar procedure, the medical instrument used in the interventional procedure may be a needle. In another example, if the interventional procedure involves a chronic total occlusion, stent placement, or other similar interventional procedure, the medical instrument may be a catheter or a guidewire.

While the doctor or the technician is moving the medical instrument towards the target of the interventional procedure, the position of the medical instrument relative to the 3-D/2-D fusion visualization 114 is displayed on the display device 116 (Block 516). In one embodiment, the monitoring device 110 uses magnetic tracking. In this embodiment, the monitoring device 110 communicates the location coordinates of the medical instrument in the organ cavity or portion of the patient to the processor 112. The processor 112 calculates the position of the medical instrument relative to the three-dimensional image dataset 108, the fusion visualization 114, and/or the two-dimensional image 106. Accordingly, the processor 112 can incorporate the position of the medical instrument in the 3-D/2-D fusion visualization 114. In another embodiment, the monitoring device 110 uses magnetic navigation, which allows the doctor or the technician to navigate the medical measurement within the organ cavity or portion of the patient. Where the doctor or the technician has registered the three-dimensional image dataset 108 to the magnetic navigation system of the monitoring device 110, the monitoring device 110 communicates the location coordinates of the medical instrument in the organ cavity or portion of the patient to the processor 112. The processor 112 calculates the position of the medical instrument relative to the three-dimensional image dataset 108, the fusion visualization 114, and/or the two-dimensional image 106. In this embodiment, the doctor or the technician can steer the medical instrument by viewing the incorporated medical instrument in the 3-D/2-D fusion visualization 114 displayed by the display device 116.

In displaying the position of the medical instrument relative to the 3-D/2-D fusion visualization 114 (Block 516), the doctor or the technician may also adjust the display mode of the medical imaging device 104 to better visualize the medical instrument. For example, the medical imaging device 104 may support a subtracted mode, which allows the processor 112 to filter unwanted noise from the 3-D/2-D fusion visualization 114. By using the subtracted mode of the medical imaging device 104, the doctor or the technician can better view the medical instrument when contrasted with the two-dimensional image 106 and the three-dimensional image representative of the three-dimensional image dataset 108 of the 3-D/2-D fusion visualization 114. Other viewing modes may also be supported by the medical imaging device 104.

After displaying the 3-D/2-D fusion visualization on the display device 116 (Block 516), the doctor or the technician may decide to update the registration of the three-dimensional image dataset 108 to the two-dimensional image 106 (Block 518). Updating the registration of the three-dimensional image dataset 108 to the two-dimensional image 106 may occur if the patient has moved during the interventional procedure or if the medical imaging device 104 has changed position or orientation of the scan region since last acquiring the two-dimensional image 106. If the doctor or the technician decides to update the registration of the three-dimensional image dataset 108 to the two-dimensional image 106, the doctor or the technician then instructs the processor 112 to update the registration of the three-dimensional image dataset 108 to the two-dimensional image 106. It is also possible that the processor 112 automatically updates the registration of the three-dimensional image dataset 108 to the two-dimensional image 106 based on input provided by the monitoring device 110 or the medical imaging device 104. In one embodiment, the update of the registration is based on motion correction. Examples of updating the registration based on motion correction include, but are not limited to, feature tracking, electrocardiogram (ECG) triggering, respiratory tracking and/or control, online registration, any other now known or later developed motion correction techniques, or combinations thereof. In one embodiment, the monitoring device 110 uses feature tracking, such as landmarks on the patient undergoing the interventional procedure, to monitor the movement of the patient. In this embodiment, the processor 112 uses the feature tracking provided by the monitoring device 110 to update the registration of the three-dimensional image dataset 108 to the two-dimensional image 106. In another embodiment, the monitoring device 110 uses ECG triggering to monitor the patient undergoing interventional procedure and provides the ECG triggering as input to the processor 112 to update the registration of the three-dimensional image dataset 108 to the two-dimensional image 106. In another embodiment, the update of the registration is based on changes in the position or orientation of the medical imaging device 104. For example, where the medical imaging devices 104 has moved between acquiring a first two-dimensional image and a second two-dimensional image, updating the registration the three-dimensional image dataset 108 may be based on the changes in the position and/or orientation of the medical imaging device 104 between the acquisition periods.

After updating the registration of the three-dimensional image dataset 108 to the two-dimensional image 106, the doctor or the technician may then verify the position of the medical instrument relative to the 3-D/2-D fusion visualization 114 (Block 522). In one embodiment, the doctor or the technician uses the monitoring device 110 to determine the location of the medical instrument in the organ cavity or portion of the patient undergoing the interventional procedure, and then compares the location of the medical instrument as reported by the monitoring device 110 with the position of the instrument as displayed in the 3-D/2-D fusion visualization 114. For example, where the monitoring device 110 uses magnetic tracking, the doctor or the technician can use the magnetic tracking features of the monitoring device 110 to determine the location of the medical instrument. In another example, where the monitoring device 110 uses magnetic navigation, the doctor or the technician can use the magnetic navigation features of the monitoring device 110 to determine the location of the medical instrument. In another embodiment, a doctor or the technician uses the medical imaging device 104 to verify the position of the medical instrument relative to the 3-D/2-D fusion visualization 114. For example, the medical imaging device 104 acquires multiple two-dimensional images from various angles, and then compares the multiple two-dimensional images with each other to confirm the location of the medical instrument. The processor 112 determines alignment by image processing, or the doctor or technician inputs data indicating proper alignment. After confirming the location of the medical instrument using the medical imaging device 104, the doctor or the technician can then compare the determined location of medical instrument with its position as displayed by the display device 116 in the 3-D/2-D fusion visualization 114. In another example, the doctor or the technician could manipulate the viewing modes supported by the medical imaging device 106 to better visualize the medical instrument in the organ cavity or portion of the patient undergoing the interventional procedure, such as where the medical imaging device 106 supports a subtracted viewing mode, to verify the location of the medical instrument.

After verifying the position of the medical instrument relative to the 3-D/2-D fusion visualization 114, the doctor or the technician, or processor 112 updates the registration of the three-dimensional image dataset 108 to the medical imaging device 104 for the monitoring device 110, depending on the device used to verify the position of the medical instrument (Block 524). For example, where the doctor or the technician used the medical imaging device 104 to verify the position of the medical measurement, the registration of the three-dimensional image dataset 108 to the two-dimensional image 106 is updated based on the geometry of the medical imaging device 104. In another example, the doctor or the technician trigger update of the registration of the three-dimensional image dataset 108 to the monitoring device 110. The processor 112 determines the spatial relationship based on sensors on the medical imaging device 104 and/or input from the monitoring device 110.

The doctor or the technician then determines whether the interventional procedure is complete (Block 526). If the interventional procedure is not complete, the display device 116 continues displaying the visualization or updates of the three-dimensional dataset 108 (Block 502). The doctor or the technician then proceeds through the acts previously described until the doctor or the technician is satisfied that the interventional procedure is complete. If the doctor or the technician determines that the interventional procedure is complete, the doctor or the technician then verifies the success of the interventional procedure (Block 528). For example, the doctor or the technician could use three-dimensional imaging techniques to verify that the interventional procedure is complete, such as 3D digital subtraction angiography, 3D digital angiography, rotational angiography, any now known or later developed three-dimensional imaging technique, or combinations thereof. Alternatively, the real-time or continuously updated 2D images are used to verify completion at the time of the procedure.

Although FIGS. 4-5 have been described with reference to a three-dimensional image dataset, it is also possible that a four-dimensional dataset is used, such as where the three-dimensional image dataset has a temporal or spatial component. One example of a three-dimensional image dataset that has a temporal component is a three-dimensional image dataset of the heart, which changes in volume size over the course of the interventional procedure. In this example, the three-dimensional image dataset of the heart with the temporal component becomes a four-dimensional image dataset. Another example of a three-dimensional image dataset that changes over time is a three-dimensional image dataset of the lungs, which also changes in volume size during the interventional procedure. In this example, the three-dimensional image dataset of the lungs with the temporal component becomes a four-dimensional image dataset. In both these examples, the heart activity or respiration activity of the four-dimensional image dataset can be registered to the two-dimensional image 106 or the magnetic tracking and/or magnetic navigation system of the monitoring device 110.

While various embodiments of the invention have been described, it will be apparent to those of ordinary skill in the art that many more embodiments and implementations are possible within the scope of the invention. Accordingly, the invention is not to be restricted except in light of the attached claims and their equivalents.

We claim:

1. A method for displaying an interventional procedure using a three-dimensional image registered to a two-dimensional image, the method comprising:
acquiring a three-dimensional image dataset representative of an organ cavity;
registering the three-dimensional image dataset to a medical imaging device;
acquiring a two-dimensional image of an interventional procedure using the medical imaging device;
performing an interventional procedure using a medical instrument; and,
displaying a representation of at least a portion of the medical instrument during the interventional procedure using a fusion visualization of the three-dimensional image dataset and the two-dimensional image.

2. The method of claim 1, where acquiring the three-dimensional image dataset representative of the organ cavity comprises acquiring the three-dimensional image dataset before the interventional procedure.

3. The method of claim 1, where acquiring the three-dimensional image dataset representative of the organ cavity comprises acquiring with an intra-operative technique.

4. The method of claim 1, where acquiring the three-dimensional image dataset comprises acquiring with an X-ray imaging device capable of acquiring three-dimensional images.

5. The method of claim 1, where acquiring the two-dimensional image comprises acquiring with an X-ray imaging device or an operation microscope.

6. The method of claim 1, further comprising:
determining a location of the medical instrument with an instrument localization device or algorithm; and
determining a position of the location relative to the three-dimensional image dataset and the two-dimensional image.

7. The method of claim 1, further comprising:
determining a location of the medical instrument with an instrument localization device or algorithm;
determining a position of the location relative to the three-dimensional image dataset and the two-dimensional image; and,
steering the medical instrument using magnetic navigation based on the position.

8. The method of claim 1, where acquiring the two-dimensional image comprises acquiring a fluoroscopic image.

9. The method of claim 1, further comprising:
dynamically updating a registration of the three-dimensional image dataset to a coordinate system of the medical imaging device.

10. The method of claim 7, where dynamically updating the registration of the three-dimensional image dataset to the medical imaging device comprises dynamically updating as a function of an electrocardiogram.

11. A system for acquiring and displaying an interventional procedure using a three-dimensional image registered to a two-dimensional image, the system comprising:
a medical imaging device operable to acquire a two-dimensional image of an organ cavity;
a monitoring device configured to monitor a medical instrument being used on the organ cavity during an interventional procedure;
a processor operable to acquire a three-dimensional image dataset representative of the organ cavity and operable to register the three-dimensional image dataset to a two-dimensional image, the two-dimensional image being representative of a scan region of the medical imaging device, the processor operable to generate a fusion visualization of the three-dimensional image dataset, the two-dimensional image, and a representation of the medical instrument as a function of an output of the monitoring device; and
a display device operable to display the fusion visualization.

12. The system of claim 11, where the three-dimensional image dataset representative of the organ cavity is acquired prior to the interventional procedure.

13. The system of claim 11, where the three-dimensional image data representative of the organ cavity is acquired during the interventional procedure.

14. The system of claim 11, where the medical imaging device is an X-ray imaging device, or an operation microscope.

15. The system of claim 11, where the monitoring device is further configured to determine a location of the medical instrument with an instrument localization device that uses magnetic tracking and the processor is further operable to determine a position of the location relative to the three-dimensional image dataset and the two-dimensional image.

16. The system of claim 11, further comprising a magnetic navigation device operative to steer the medical instrument based on a location of the medical instrument and a position of the location relative to the three-dimensional image dataset and the two-dimensional image, where
the monitoring device is further configured to determine the location of the medical instrument; and,
the processor is further operative to determine the position of the location relative to the three-dimensional image dataset and the two-dimensional image.

17. The system of claim 11, where the two-dimensional image is a fluoroscopic image.

18. The system of claim 11, where the processor is further operable to dynamically update the registration of the three-dimensional image dataset to a coordinate system of the medical imaging device.

19. The system of claim 18, where the processor dynamically updates the registration of the three-dimensional image dataset to the medical imaging device based on an output of an electrocardiogram.

20. A computer-readable medium having computer-executable instructions for performing a method, the method comprising:
acquiring a three-dimensional image dataset representative of an organ cavity;
registering the three-dimensional image dataset to a medical imaging device;
acquiring a two-dimensional image of an interventional procedure using the medical imaging device;
performing the interventional procedure on the organ cavity using a medical instrument; and,
displaying a representation of at least a portion of the medical instrument during the interventional procedure using a fusion visualization of the three-dimensional image dataset and the two-dimensional image.

21. The computer-readable medium of claim 20, where acquiring the three-dimensional image dataset representative of the organ cavity comprises acquiring the three-dimensional image dataset before the interventional procedure.

22. The computer-readable medium of claim 20, where acquiring the three-dimensional image dataset representative of the organ cavity comprises acquiring with an intra-operative technique.

23. The computer-readable medium of claim 20, where the medical imaging device is an X-ray imaging device, or an operation microscope.

24. The computer-readable medium of claim 20, further comprising computer-executable instructions to determine a location of the medical instrument with an instrument localization device and to determine a position of the location relative to the three-dimensional image dataset and the two-dimensional image.

25. The computer-readable medium of claim 20, further comprising computer-executable instructions to determine a location of the medical instrument with an instrument localization device, to determine a position of the location relative to the three-dimensional image dataset and the two-dimensional image, and to steer the medical instrument using magnetic navigation based on the position.

26. The computer-readable medium of claim 20, where acquiring the two-dimensional image comprises acquiring fluoroscopic images.

27. The computer-readable medium of claim 20, further comprising computer-executable instructions to dynamically update the registration of the three-dimensional image dataset to a coordinate system of the medical imaging device.

28. The computer-readable medium of claim 27, where dynamically updating the registration of the three-dimensional image dataset to the medical imaging device comprises dynamically updating as a function of an electrocardiogram.
